(19) United States
(12) Patent Application Publication
(10) Pub. No.: US 2020/0107713 A1
(43) Pub. Date: Apr. 9, 2020

(54) ARTHROSCOPIC INSTRUMENT FOR IMPROVED VIEWING OF GUTTER OR OFFSETTING OF K-WIRE

(71) Applicant: CONMED CORPORATION, Utica, NY (US)

(72) Inventors: Matthew Summitt, Palm Harbor, FL (US); Robert Thibodeau, St. Petersburg, FL (US)

(73) Assignee: CONMED CORPORATION, Utica, NY (US)

(21) Appl. No.: 16/595,779

(22) Filed: Oct. 8, 2019

Related U.S. Application Data
(60) Provisional application No. 62/742,724, filed on Oct. 8, 2018.
(60) Provisional application No. 62/742,742, filed on Oct. 8, 2018.

(51) Int. Cl. A61B 1/317; A61B 17/00

(52) U.S. Cl. CPC A61B 1/317; A61B 17/00234

(57) ABSTRACT

A polished surface of an arthroscopic instrument for improving visualization of the portion of an ankle joint under repair known as the gutter. The instrument has a handle, a shaft, and a head, at least a portion of the head is polished to provide a mirrored surface for viewing the gutter. Alternatively, the arthroscopic instrument may have a handle, a shaft, and a head, where the head is cylindrical and includes a plurality of apertures extending along the axis of the cylindrical head to define a plurality of guides.

### CROSS-REFERENCE TO RELATED APPLICATIONS

The present application claims priority to U.S. Provisional No. 62/742,724, filed on Oct. 8, 2018 and to Provisional No. 62/742,742, filed on Oct. 8, 2018.

### BACKGROUND OF THE INVENTION

### 1. Field of the Invention

The present invention relates to arthroscopic instruments and, more specifically, to an approach for improving visualization and offsetting guide wires.

### 2. Description of the Related Art

In arthroscopic surgeries, such as those involving the ankle, it is often difficult for the surgeon to visualize the portion of the joint known as the gutter. In arthroscopic surgeries, there is a need for a surgeon to place a k-wire offset from a k-wire that has already been drilled into a bone. Accordingly, there is a need in the art for an instrument that can help visualize a gutter or to place an off-set k-wire.

### BRIEF SUMMARY OF THE INVENTION

In a first embodiment, the present invention is a polished surface of an arthroscopic instrument for improving visualization of the portion of a joint under repair known as the gutter. More specifically, in a device having a handle, a shaft, and a head, at least a portion of the head is polished to provide a mirrored surface for viewing the gutter. The head is oriented at an angle relative to the longitudinal axis of the shaft so that the mirrored surface provides a sight line that extends obliquely to the longitudinal axis. The invention may therefore include a method of viewing a portion of a joint during an arthroscopic procedure. One step of the method involves providing an instrument having a handle, a shaft extending from the handle, and a head mounted to the end of the shaft, wherein at least a portion of the head includes a region having a mirrored surface that extends obliquely to the longitudinal. Another step of the method involves the step of positioning the instrument so that the region having the mirrored surface reflects the portion of the joint to be viewed.

In another embodiment, the present invention is an arthroscopic instrument a handle, a shaft, and a head, the head is cylindrical and includes a plurality of apertures extending along the axis of the cylindrical head to define a plurality of guides. Kirschner wires (k-wires) may be affixed to a site using the apertures to define and control the placement of at least a second k-wire offset from a previously installed k-wire. The invention therefore also includes a method of offsetting a subsequent k-wire from a previously installed k-wire. The method involves the step of positioning a device having a handle, a shaft extending from the handle, a head mounted to the end of the shaft and extending cylindrically along an axis, and a plurality of apertures formed through the head and extending in parallel to the axis of head so that an installed k-wire extending through one of the plurality of apertures. The method also involves installing a different k-wire using another of the plurality of apertures as a guide to offset the different k-wire from the installed k-wire.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of an arthroscopic instrument having a gutter mirror according to the present invention; and

FIG. 2 is a schematic of an arthroscopic instrument having a guide for offsetting a k-wire according to the present invention.

### DETAILED DESCRIPTION OF THE INVENTION

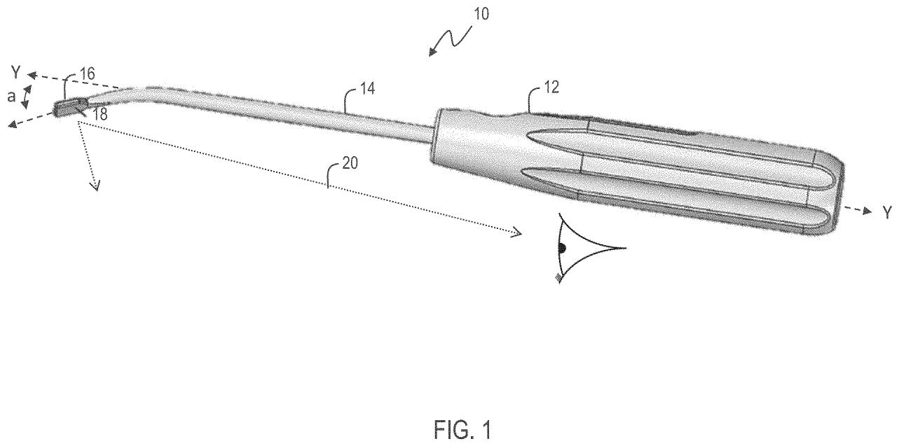

Referring to the figures, wherein like numeral refer to like parts throughout, there is seen in FIG. 1 an arthroscopic instrument 10 for improving visualization of the portion of a joint under repair known as the gutter. More specifically, arthroscopic instrument 10 includes a handle 12, a shaft 14, and a head 16, where a region 18 of head 16 is polished to provide a mirrored surface. Head 16 is oriented at an oblique angle a relative to a longitudinal axis Y-Y of the shaft such that region 18 having the mirrored surface will provide a sight line 20 that is angled obliquely relative to the longitudinal axis Y-Y. The mirrored surface thus reflects sight line 20 extending along the shaft obliquely to the longitudinal axis. It should be recognized that sight line is likely provided by an arthroscopy system. Arthroscopic instrument 10 may be inserted into a joint under repair and manipulated to allow a user to view, using the arthroscopy system, the gutter of the joint via reflection provided by region 18 of head 16.

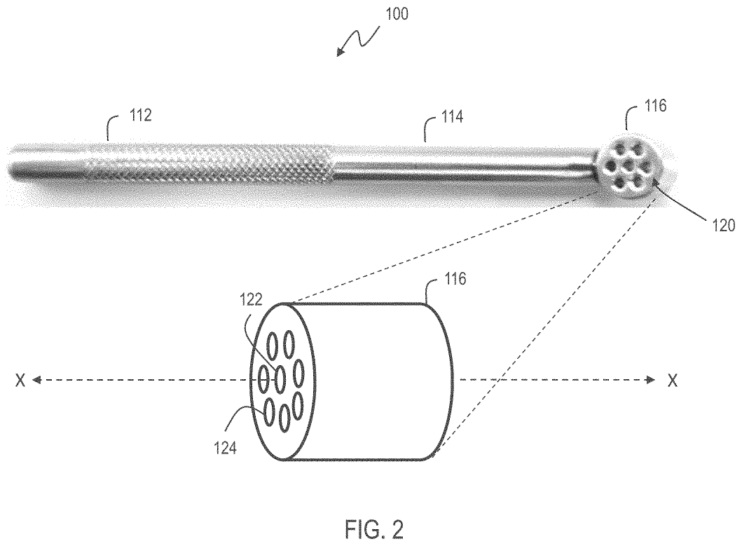

Referring to FIG. 2, an arthroscopic instrument 100 for offsetting a k-wire comprises a handle 112, a shaft 114, and a head 116. Head 116 is cylindrical and includes a series of apertures 120 extending in parallel to the cylindrical axis of head 116 and each other. Apertures 120 are arranged to include a central aperture 122 surrounded by a series of circumferentially positioned apertures 124. It should be recognized that the geometric arrangement of apertures 120 may be varied as desired. Apertures 120 may be used to allow a subsequent Kirschner wires (k-wire) to be installed in a location a predetermined offset from a prior installed k-wire by positioning head 116 over the first k-wire so that the k-wire extending through one of apertures 120 and then using another aperture 120 as a guide for the installation of another k-wire. The offsetting and geometric arrangement of apertures 120 may thus be used to control offsetting of additional k-wires once a first k-wire has been affixed at a site. For example, arthroscopic instrument 100 is positioned so that an installed k-wire extends through one of the plurality of apertures. A different k-wire may then be installed using another of the plurality of apertures as a guide to offset the different k-wire from the installed k-wire.

What is claimed is:

1. A method of viewing a gutter of a joint during an arthroscopic procedure, comprising the steps of:
providing an instrument having a handle, a shaft extending from the handle, and a head mounted to an end of the shaft, wherein at least a portion of the head includes a region having a mirrored surface that is positioned at an oblique angle relative to a longitudinal axis of the head; and
positioning the instrument so that the region having the mirrored surface reflects the portion of the joint to be viewed.

2. The method of claim 1, wherein the shaft includes a bend proximate to the head so that head is oriented to produce the oblique angle relative to the longitudinal axis of the shaft.

3. The method of claim 2, wherein the portion of the head including the region is on a side of the head that faces the handle.

4. The method of claim 3, wherein the mirrored surface is positioned to reflect a sight line extending along the longitudinal axis of the shaft obliquely to the longitudinal axis.

5. An arthroscopic instrument, comprising:
a handle;
a shaft extending from the handle;
a head mounted to an end of the shaft and extending cylindrically along an axis; and
a plurality of apertures formed through the head and extending in parallel to the axis of the head.

6. The arthroscopic instrument of claim 5, wherein the plurality of apertures includes a single, centrally positioned aperture.

7. The arthroscopic instrument of claim 6, wherein the remaining plurality of apertures are positioned around the single, centrally positioned.

8. A method of offsetting a subsequent k-wire from a previously installed k-wire, comprising the step of:
positioning a device having a handle, a shaft extending from the handle, a head mounted to the end of the shaft and extending cylindrically along an axis, and a plurality of apertures formed through the head and extending in parallel to the axis of the head so that an installed k-wire extends through one of the plurality of apertures; and
installing a different k-wire using another of the plurality of apertures as a guide to offset the different k-wire from the installed k-wire.

9. The method claim 8, wherein the plurality of apertures includes a single, centrally positioned aperture.

10. The method claim 9, wherein any remaining of the plurality of apertures are positioned around the single, centrally positioned aperture.
